(19) United States
(12) Patent Application Publication
(10) Pub. No.: US 2025/0064588 A1
(43) Pub. Date: Feb. 27, 2025

(54) CONTROLLED OPENING OF EXPANDABLE ACCESS SHEATHS

(71) Applicant: EDWARDS LIFESCIENCES CORPORATION, Irvine, CA (US)

(72) Inventors: Michael G. Valdez, Riverside, CA (US); Omar Fawzi Azanki, Coto de Caza, CA (US); Hien T. Nguyen, Aliso Viejo, CA (US)

(21) Appl. No.: 18/943,635

(22) Filed: Nov. 11, 2024

Related U.S. Application Data
(63) Continuation of application No. PCT/US2023/021786, filed on May 10, 2023.
(60) Provisional application No. 63/341,750, filed on May 13, 2022.

(51) Int. Cl. A61F 2/24

(52) U.S. Cl. CPC A61F 2/2436

(57) ABSTRACT

A medical access system can comprise an expandable access sheath and an introducer. The expandable access sheath can comprise a sheath shaft comprising an expandable distal end portion, and a shaft delivery lumen extending therethrough. The introducer can comprise an introducer shaft configured to be at least partially disposed within the shaft delivery lumen. The introducer shaft can comprise a first distal portion comprising a first diameter and a second distal portion proximal of the first distal portion and having a second diameter larger than the first diameter. A second externally oriented surface portion defining the second distal portion, and an externally oriented transition surface portion extending between a first externally oriented surface portion defining the first distal portion and the second externally oriented surface, can be angled relative to one another, such as forming an angle between about 80 degrees and about 100 degrees.

### CROSS REFERENCE TO RELATED APPLICATIONS

This application is a continuation of International Patent Application No. PCT/US2023/021786, filed May 10, 2023, which claims the benefit of U.S. Provisional Patent Application No. 63/341,750, filed May 13, 2022, the complete disclosures of which are hereby expressly incorporated by reference herein in their entireties for all purposes.

### BACKGROUND

The present disclosure generally relates to the field of minimally invasive transcatheter delivery of medical devices. Minimally invasive transcatheter medical procedures can comprise use of expandable medical sheaths for delivery of medical devices. Minimally invasive transcatheter procedures can be used to treat a variety of conditions, including conditions of the heart.

### SUMMARY

Described herein are methods and devices related to medical access systems that comprise an expandable access sheath and an introducer configured to be preloaded within the expandable access sheath. The expandable access sheath can comprise a sheath shaft with an expandable distal end portion. The introducer can be preloaded within the expandable access sheath while the expandable distal end portion is in a closed state. The introducer can be translated distally relative to the expandable access sheath while disposed therein such that one or more portions of the introducer can be pushed against corresponding portions of the expandable distal end portion to cause transformation of the expandable distal end portion from the closed state to an open state.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows a medical access system disposed within a femoral artery for minimally invasive transcatheter delivery of a medical device to a target heart location via an access opening on the femoral artery in accordance with one or more examples.

FIG. 2A provides a longitudinal cross-sectional view of the medical access system described with reference to FIG. 1, where an introducer is preloaded within an expandable access sheath in accordance with one or more examples.

FIG. 2B provides a longitudinal cross-sectional view of the medical access system of FIG. 2A showing the introducer in a deployed position relative to the expandable access sheath in accordance with one or more examples.

FIG. 2C provides a perspective view of the introducer of FIG. 2A in accordance with one or more examples.

FIG. 2D provides a perspective view of the medical access system of FIG. 2A in accordance with one or more examples.

FIG. 3A provides a longitudinal cross-sectional view of a medical access system comprising a spacer member configured to maintain an introducer in a preloaded position within an expandable access sheath in accordance with one or more examples.

FIG. 3B provides a longitudinal cross-sectional view of the medical access system of FIG. 3A showing the spacer member removed and the introducer in a deployed position relative to the expandable access sheath in accordance with one or more examples.

FIG. 4A provides a cross-sectional view of a medical access system that includes an introducer with an introducer shaft that comprises a second distal portion and a portion proximal of the second distal portion having a third diameter smaller than that of the second distal portion in accordance with one or more examples.

FIG. 4B provides a perspective view of the introducer of FIG. 4A in accordance with one or more examples.

FIGS. 5A and 5B provide longitudinal cross-sectional and perspective views, respectively, of a medical access system comprising an introducer with a first and second partial rigid circumferential portion disposed around respective portions of a circumference of an introducer shaft in accordance with one or more examples.

FIG. 6 provides a longitudinal cross-sectional view of a medical access system having an introducer in a preloaded position within an expandable access sheath in accordance with one or more examples.

FIG. 7 provides a longitudinal cross-sectional view of a medical access system having an introducer in a preloaded position within an expandable access sheath in accordance with one or more examples.

FIG. 8 provides a longitudinal cross-sectional view of a medical access system comprising an introducer in a deployed position relative to an expandable access sheath in accordance with one or more examples.

FIG. 9 provides a longitudinal cross-sectional view of a medical access system comprising an introducer in a deployed position relative to an expandable access sheath in accordance with one or more examples.

FIG. 10 is a process flow diagram illustrating a process for delivering a medical device to a target location using a medical access system as described herein in accordance with one or more examples.

### DETAILED DESCRIPTION

The headings provided herein are for convenience only and do not necessarily affect the scope or meaning of the claimed invention.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for case of description to describe the positional relationship between element(s)/structures(s), as illustrated in the drawings. It should be understood that spatially relative terms are intended to encompass different orientations of the clement(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Expandable access sheaths comprising expandable distal end portions can be used in minimally invasive procedures to facilitate transcatheter delivery of medical devices. An expandable access sheath comprising an expandable distal end portion can be inserted into a vessel and/or lumen prior to advancement therethrough of a medical device or a delivery catheter carrying the medical device. The expandable access sheath can be inserted into the vessel and/or lumen while the expandable distal end portion is in a closed state. The expandable distal end portion of the sheath can transform from the closed state to an open state to allow passage therethrough of the delivery catheter and/or medical device. Achieving desired opening of the expandable distal end portion without damaging the expandable distal end portion and/or surrounding tissue can be difficult.

Systems, devices, and methods described herein relate to medical access systems configured to facilitate minimally invasive transcatheter delivery of medical devices to any number of target locations. A medical access system described herein can comprise an expandable access sheath comprising a sheath shaft with an expandable distal end portion. The medical access system can comprise an introducer configured to be preloaded within the expandable access sheath while the expandable distal end portion is in a closed state. The sheath shaft can comprise a shaft delivery lumen. The introducer can comprise an introducer shaft comprising a portion configured to be slidably received and/or disposed within the shaft delivery lumen. The introducer can be translated distally relative to the expandable access sheath to cause transformation of the expandable distal end portion from the closed state to an open state. For example, one or more portions of the introducer can be used to push against corresponding portions of an inner lumen wall defining the shaft delivery lumen extending through the expandable distal end portion. Pressing the introducer against the inner lumen wall can cause the expandable distal end portion to expand, unfold and/or open. In some instances, the expandable distal end portion can comprise a plurality of pleats. The plurality of pleats can be bonded together to maintain the expandable distal end portion in the closed state. Pushing the introducer against the inner lumen wall of the expandable distal end portion can break the bonding of the pleats, thereby allowing the plurality of pleats to unfold and assume an open configuration.

In some instances, a distal portion of the introducer shaft can comprise a first distal portion with a portion having a first diameter and a second distal portion proximal of the first distal portion and having a second diameter. The second diameter can be larger than the first diameter such that one or more portions of a distal end of the second distal portion can be laterally disposed relative to the first distal portion. The laterally disposed portions of the distal end can facilitate engagement of the one or more portions of the distal end with corresponding portions of an inner lumen wall of the shaft delivery lumen. The second distal portion can be disposed within the shaft delivery lumen while the expandable distal end portion is closed. One or more portions of the second distal portion can be configured to frictionally contact and/or engage with corresponding portions of the inner lumen wall defining the shaft delivery lumen. Frictional contact and/or engagement between the second distal portion and the inner lumen wall can facilitate maintaining the position of introducer, such as while the introducer is preloaded within the expandable access sheath. The introducer can be deployed such that the distal end of the second distal portion can be pushed against the corresponding portions of the inner lumen wall defining the shaft delivery lumen extending through the expandable distal end portion.

It will be understood that portions of a sheath shaft proximal of an expandable distal end portion as described herein can comprise portions which are expandable or nonexpendable. In some instances, the sheath shaft proximal of the expandable distal end portion can be non-expandable, maintaining the same or similar width. In some instances, the sheath shaft proximal of the expandable distal end portion can be expandable. For example, portions of the sheath shaft proximal of the expandable distal end portion can reversibly expand radially to assume configurations of various widths. In some instances, a portion of the sheath shaft proximal of the expandable distal end portion can be expandable and another portion of the sheath shaft proximal of the expandable distal end portion can be non-expandable.

Although the medical access systems described herein are described as being inserted into the femoral artery for delivery of medical devices to the heart, it will be understood that the medical access systems can be inserted into any number of other vessels and/or lumens to facilitate delivery of medical devices to any number of other organs, chambers, vessels and/or lumens. In some instances, the medical access systems can be inserted into another arterial access opening. In some instances, a venous access approach can be used. For example, the medical access systems can be inserted into a femoral vein, internal jugular vein, and/or subclavian vein. The medical access systems can be used in any number of different types of minimally invasive transcatheter procedures.

It will be understood that one or more components of the medical access systems can undergo various processes in preparation for their use in the procedures, including for example sterilization processes. The medical access systems can be sterilized medical access systems. For example, expandable access sheaths and/or introducers as described herein can be sterilized expandable access sheaths and/or sterilized introducers. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

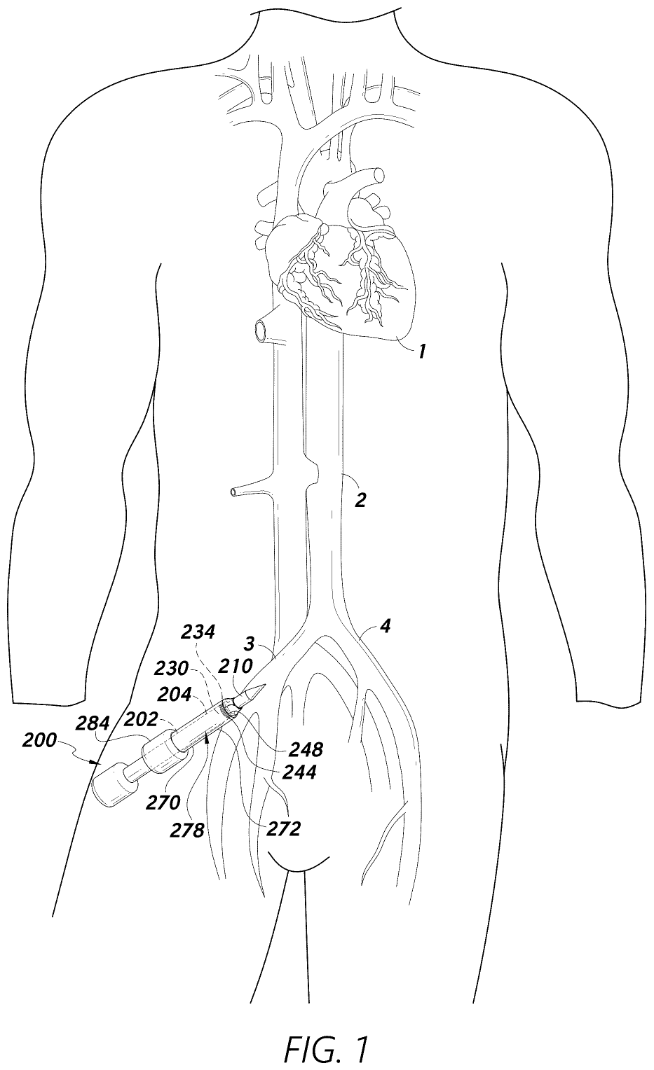

FIG. 1 shows an example of a medical access system 200 comprising an introducer 202 preloaded within an expandable access sheath 270, where the expandable access sheath 270 carrying the introducer 202 comprises a portion disposed within a femoral artery 3. The introducer 202 can comprise an introducer shaft 204 extending distally from an introducer hub 260. The expandable access sheath 270 can comprise a sheath shaft 272 comprising a shaft delivery lumen 278 extending therethrough, and extending distally from a sheath hub 284. The sheath shaft 272 can comprise an expandable distal end portion 276. While the sheath shaft 272 is inserted into the right femoral artery 3 through an access opening formed thereon and advanced to a target location, the expandable distal end portion 276 can be in a closed state. A portion of the introducer shaft 204 can be disposed within the shaft delivery lumen 278 while at least a portion of the sheath shaft 272 is inserted into the right femoral artery 3. The sheath shaft 272 carrying the introducer shaft 204 can be advanced to a target position, such as a target position within the right femoral artery 3. It will be understood that the location of the access opening is shown for illustrative purposes only. The access opening can be made on any number of other vessels and/or lumens of the patient, including for example, the left femoral artery 4. In some instances, the access opening can be made on another arterial vessel. In some instances, a venous access approach can be used. For example, the access opening can be made on a femoral vein, internal jugular vein, or subclavian vein.

While the introducer 202 is preloaded within the expandable access sheath 270, a second distal portion 230 of the introducer shaft 204 can be disposed within the shaft delivery lumen 278. A first distal portion 210 of the introducer shaft 204 distal of the second distal portion 230 can comprise a portion disposed within the shaft delivery lumen 278 and a portion disposed distally of the sheath shaft 272. The introducer 202 can be translated distally relative to the expandable access sheath 270 to transform the expandable distal end portion 276 from the closed state to an open state. A distal end 234 of the second distal portion 230 can be pushed against the expandable distal end portion 276 to cause the expandable distal end portion 276 to expand and/or unfold. For example, the distal end 234 of the second distal portion 230 can be pushed against an inner lumen wall defining the shaft delivery lumen 278 extending through the expandable distal end portion 276 to cause the expandable distal end portion 276 to transform from the closed state to the open state.

The introducer 202 can be retracted and withdrawn from the expandable access sheath 270 after the expandable distal end portion 276 has been opened. The expandable access sheath 270 can be subsequently used for minimally invasive transcatheter delivery of a medical device. The medical device or a delivery catheter carrying the medical device can be advanced through the expandable access sheath 270 to a target location. In some instances, the target location can be a target heart location. For example, the medical device can be advanced through the expandable access sheath 270 positioned within the right femoral artery 3 and into the aorta 2. In some instances, the medical device can be delivered into the heart 1 from the aorta 2. It will be understood that the delivery of the medical device to the heart 1 through the right femoral artery 3 and aorta 2 is an illustrative purposes only. The expandable access sheath 270 can be inserted into any number of other vessels and/or lumens for minimally invasive transcatheter delivery of a medical device to another target location via other delivery pathways.

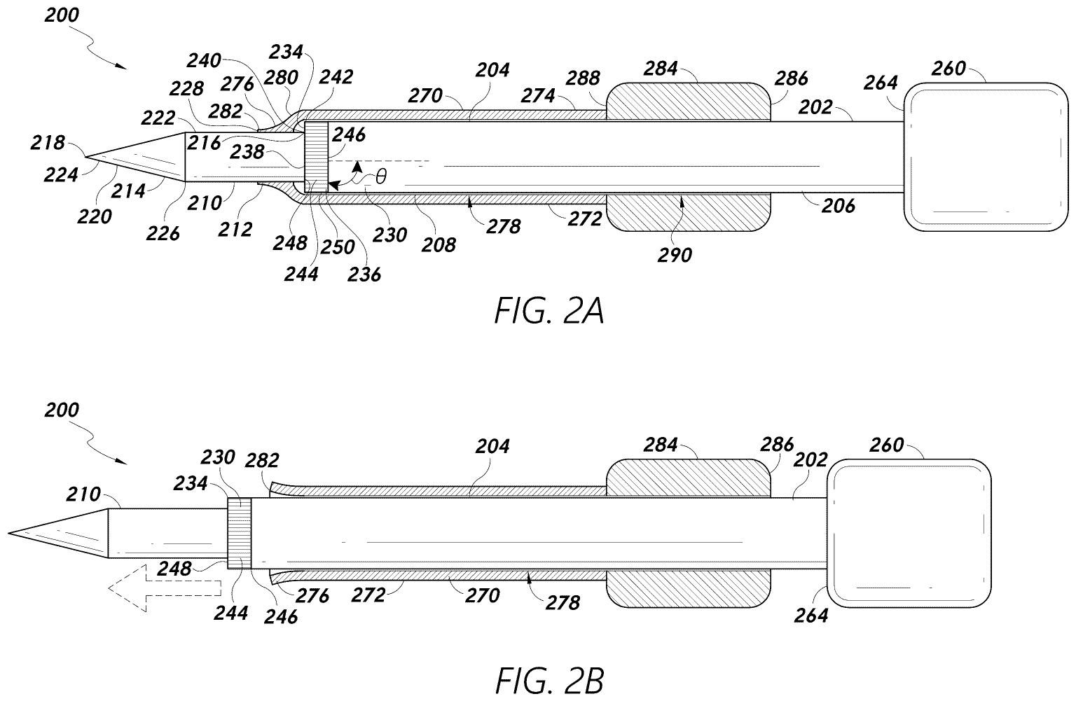

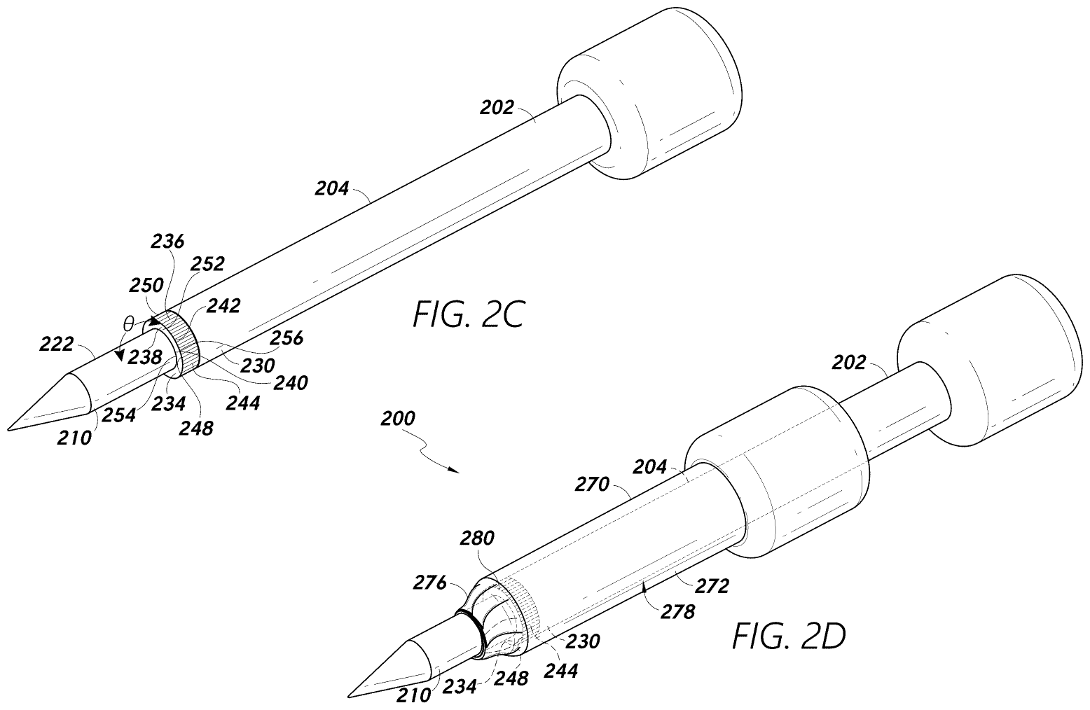

FIGS. 2A through 2D are various views showing in further detail the medical access system 200 described with reference to FIG. 1. As described herein, the medical access system 200 can comprise an introducer 202 and an expandable access sheath 270. The expandable access sheath 270 can comprise a sheath shaft 272 comprising a shaft delivery lumen 278 extending therethrough. The sheath shaft 272 can comprise an expandable distal end portion 276. The expandable distal end portion 276 can be configured to assume an open configuration or a closed configuration. In some instances, the expandable distal end portion 276 in the closed state can be configured for atraumatic navigation. FIG. 2A is a side cross-sectional view of the medical access system 200 while the introducer 202 is in a preloaded position within the expandable access sheath 270 and the expandable distal end portion 276 of the expandable access sheath 270 is in a closed state. The introducer 202 can comprise an introducer shaft 204. A distal portion 208 of the introducer shaft 204 can comprise a first distal portion 210 with a portion having a first lateral dimension, such as a first diameter, and a second distal portion 230 having a second lateral dimension, such as a second diameter. A lateral dimension, such as a diameter, can be a dimension perpendicular or substantially perpendicular to a longitudinal axis of the introducer shaft 204. The second distal portion 230 can be proximal of the first distal portion 210. The second diameter can be larger than the first diameter such that one or more portions of a distal end 234 of the second distal portion 230 can be laterally disposed relative to the first distal portion 210. The laterally disposed portions of the distal end 234 can facilitate engagement of the one or more portions of the distal end 234 with corresponding portions of an inner lumen wall of the shaft delivery lumen 278. For example, while the introducer 202 is preloaded within the expandable access sheath 270, the second distal portion 230 can be disposed within the shaft delivery lumen 278. The distal end 234 of the second distal portion 230 can be disposed within the shaft delivery lumen 278. One or more portions of the second distal portion 230 can be configured to contact and/or engage with corresponding portions of an inner lumen wall defining the shaft delivery lumen 278 extending through the expandable distal end portion 276 so as to facilitate transformation of the expandable distal end portion 276 from the closed state to an open state and/or maintain the introducer 202 in the preloaded position. As described in further detail herein, the introducer 202 can be deployed such that the distal end 234 of the second distal portion 230 can be pushed against the corresponding portions of the inner lumen wall defining the shaft delivery lumen 278 extending through the expandable distal end portion 276. Pushing the distal end 234 against the inner lumen wall can cause the expandable distal end portion 276 to open such that the expandable distal end portion 276 can assume the open state. FIG. 2B is a side cross-sectional view of the medical access system 200 while the expandable distal end portion 276 is in the open state and the introducer 202 is in a deployed state. FIG. 2C is a perspective view of the introducer 202 and FIG. 2D is a perspective view of the medical access system 200 comprising the introducer 202 preloaded within the expandable access sheath 270.

Referring to FIG. 2A, the first distal portion 210 can comprise a portion having the first diameter. The first distal portion 210 can comprise a first externally oriented surface portion 222 defining the portion of the first distal portion 210 having the first diameter. In some instances, the first externally oriented surface portion 222 can extend along a proximal portion 212 of the first distal portion 210. For example, the first externally oriented surface portion 222 can extend circumferentially around the longitudinal axis of the introducer shaft 204 to define the proximal portion 212 of the first distal portion 210. In some instances, the first externally oriented surface portion 222 can be parallel or substantially parallel to the longitudinal axis of the introducer shaft 204. For example, the first externally oriented surface portion 222 can be a circumferential portion forming a cylindrical or substantially cylindrical shape.

The first distal portion 210 can comprise a tapered portion 220. The tapered portion 220 can comprise a taper along a direction extending from a proximal end 216 to a distal end 218 of the first distal portion 210. The distal end 218 of the first distal portion 210 can be a distal end of the introducer shaft 204. In some instances, the tapered portion 220 can taper to the distal end 218 of the first distal portion 210. For example, the first distal portion 210 can comprise a tapered externally oriented surface portion 224 defining the tapered portion 220. The tapered externally oriented surface portion 224 can define a portion of the introducer shaft 204 with a decreasing diameter along a direction extending from the proximal end 216 to the distal end 218 of the first distal portion 210, such as to form a tapered profile. In some instances, the tapered portion 220 can extend along a distal portion 214 of the first distal portion 210. In some instances, the tapered portion 220 can be distal of and adjacent to the portion of the first distal portion 210 comprising the first diameter. In some instances, the first externally oriented surface portion 222 can extend from a proximal end 216 of the first distal portion 210. In some instances, the first externally oriented surface portion 222 can extend from a proximal end 216 of the first distal portion 210 to a proximal end 226 of the tapered externally oriented surface portion 224. Alternatively, an introducer shaft can comprise one or more intermediate portions between its tapered portion and first distal portion.

The tapered portion 220 can be configured to facilitate navigation of the introducer 202 and the expandable access sheath 270 through anatomical pathways. The introducer 202 can be preloaded within the expandable access sheath 270 while the expandable access sheath 270 is advanced through an anatomical pathway to a target position. For example, while the expandable distal end portion 276 is in the closed state and advanced through the anatomical pathway, at least a portion of the first distal portion 210 can be disposed distally of a distal end of the sheath shaft 272. In some instances, at least a portion of the tapered portion 220 can be disposed distally of a distal end 282 of the expandable distal end portion 276 of the sheath shaft 272. The distal end 282 of the expandable distal end portion 276 can be a distal end of the sheath shaft 272. In some instances, the entire tapered portion 220 can be disposed distally of a distal end 282 of the expandable distal end portion 276. A portion of the first distal portion 210 can be disposed through a distal opening 228 at the distal end 282 of the expandable distal end portion 276, for example at the distal end of the sheath shaft 272, such that at least a portion of the tapered portion 220 is positioned distally of the sheath shaft 272. In some instances, the portion of the first distal portion 210 having the first diameter can be disposed through the distal opening 228 such that the entire tapered portion 220 is disposed distally of the sheath shaft 272.

As described herein, while the expandable distal end portion 276 is in the closed state, the second distal portion 230 can be configured to be disposed within the shaft delivery lumen 278. In some instances, the distal end 234 of the second distal portion 230 can be at or proximate to a proximal end 280 of the expandable distal end portion 276. For example, while the introducer 202 is preloaded within the expandable access sheath 270, the distal end 234 of the second distal portion 230 can be disposed at a position within the shaft delivery lumen 278 at the proximal end 280 of the expandable distal end portion 276. The distal end 234 can be in contact and/or be engaged with one or more portions of an inner lumen wall defining the shaft delivery lumen 278. In some instances, at least a portion of the distal end 234 of the second distal portion 230 can frictionally fit against a respective inner lumen wall surface portion defining the shaft delivery lumen 278 extending through the expandable distal end portion 276. For example, the distal end 234 of the second distal portion 230 can frictionally fit against an inner lumen wall surface portion defining the shaft delivery lumen 278 at or proximate to the proximal end 280 of the expandable distal end portion 276. Frictional fit between the distal end 234 of the second distal portion 230 and the inner lumen wall can facilitate maintaining the introducer 202 at a desired position within the expandable access sheath 270.

Referring again to FIG. 2A, the second distal portion 230 can comprise at least a portion having the second diameter. In some instances, the first diameter and the second diameter can have a ratio between about 1:3 and about 2:3. In some instances, the first diameter and the second diameter have a ratio of about 1:2. A second externally oriented surface portion 236 of the introducer shaft 204 can define the portion of the second distal portion 230 having the second diameter. The second externally oriented surface portion 236 can extend circumferentially around the longitudinal axis of the introducer shaft 204. In some instances, the second distal portion 230 can have the second diameter along an entire length of the second distal portion 230. The second externally oriented surface portion 236 of the introducer shaft 204 can extend along an entire length of the second distal portion 230. The second externally oriented surface portion 236 can be a circumferential surface portion extending along an entire length of the second distal portion 230. In some instances, the second externally oriented surface portion 236 can be parallel or substantially parallel to a longitudinal axis of the introducer shaft 204. For example, the second externally oriented surface portion 236 can be a circumferential portion defining a second distal portion 230 forming a cylindrical or substantially cylindrical shape.

In some instances, at least a portion of the distal end 234 of the second distal portion 230 is rigid. The rigidity of the distal end 234 can facilitate desired contact and/or engagement with the inner lumen wall defining the shaft delivery lumen 278. In some instances, the second distal portion 230 can comprise a rigid circumferential portion 244. The rigid circumferential portion 244 can form a portion of the second distal portion 230. A distal end 248 of the rigid circumferential portion 244 can be the distal end 234 of the second distal portion 230. An externally oriented lateral surface 250 of the rigid circumferential portion 244 can form at least a portion of the second externally oriented surface portion 236. For example, the externally oriented lateral surface 250 can extend circumferentially around the longitudinal axis of the introducer shaft 204. The externally oriented lateral surface 250 can define a cylindrical or substantially cylindrical shape. In some instances, the second distal portion 230 can comprise a proximal end 246 of the rigid circumferential portion 244. The rigid circumferential portion 244 can have a ring configuration. The rigid circumferential portion 244 can have the second diameter. In some instances, the rigid circumferential portion 244 can comprise a metallic material. In some instances, the rigid circumferential portion 244 can be a metallic ring.

An externally oriented transition surface portion 238 of the introducer shaft 204 can extend between the first externally oriented surface portion 222 defining the portion of the first distal portion 210 having the first diameter and the second externally oriented surface portion 236 defining the portion of the second distal portion 230 having the second diameter. In some instances, the externally oriented transition surface portion 238 can extend from the proximal end 216 of the first distal portion 210 to the second externally oriented surface portion 236 of the second distal portion 230. In some instances, a first edge 240 of the externally oriented transition surface portion 238 can be adjacent to and in contact with the first externally oriented surface portion 222. A second edge 242 of the externally oriented transition surface portion 238 can be adjacent to and in contact with the second externally oriented surface portion 236. For example, the externally oriented transition surface portion 238 can extend from the first externally oriented surface portion 222 to the second externally oriented surface portion 236.

In some instances, the externally oriented transition surface portion 238 and the second externally oriented surface portion 236 can be angled relative to one another, such as forming an angle between about 60° and about 120°, including about 80° and about 100°. In some instances, the externally oriented transition surface portion 238 and the second externally oriented surface portion 236 can form an angle between about 85° and about 95°. In some instances, the externally oriented transition surface portion 238 is perpendicular or substantially perpendicular to the second externally oriented surface portion 236. As described herein, the second externally oriented surface portion 236 can be parallel or substantially parallel to a longitudinal axis of the introducer shaft 204. The angle “Θ” in FIG. 2A labels an angle formed by the externally oriented transition surface portion 238 and the longitudinal axis of the introducer shaft 204, and can have one or more values described herein, including for example, between about 60° and about 120°, between about 80° and about 100°, and between about 85° and about 95°, including about 90°.

In some instances, the remaining introducer shaft 204 proximal of the second distal portion 230 comprises the second diameter. In some instances, the remaining introducer shaft 204 proximal of the rigid circumferential portion 244 comprises the second diameter. For example, portions of the introducer shaft 204 proximal of the distal end 234 of the second distal portion 230 can have a uniform or substantially uniform diameter.

A proximal portion 206 of the introducer shaft 204 can be associated with an introducer hub 260. For example, the introducer shaft 204 can extend distally from the introducer hub 260, including from a distal end 264 of the introducer hub 260. The proximal portion 206 of the introducer shaft 204 can be coupled to the introducer hub 260, including the distal end 264 of the introducer hub 260. The sheath shaft 272 of the expandable access sheath 270 can extend distally from a sheath hub 284. A proximal portion 274 of the sheath shaft 272 can be associated with a sheath hub 284, including a distal end 288 of the sheath hub 284. The shaft delivery lumen 278 can be in fluid communication with a hub delivery lumen 290 extending through the sheath hub 284. For example, while the introducer 202 is preloaded within the expandable access sheath 270, a portion of the introducer shaft 204 can be disposed through the hub delivery lumen 290 and the shaft delivery lumen 278. At least a portion of the proximal portion 206 of the introducer shaft 204 can extend proximally from a proximal end 286 of the sheath hub 284. The introducer hub 260 can be disposed proximally of and spaced from the proximal end 286 of the sheath hub 284.

Referring to FIG. 2B, the introducer 202 can be configured to be translated distally relative to expandable access sheath 270 to transform the expandable distal end portion 276 from a closed state to an open state. Portions of the introducer shaft 204 disposed within the shaft delivery lumen 278 can be translated distally within relative to the sheath shaft 272. The distal end 234 of the second distal portion 230, including the distal end 248 of the rigid circumferential portion 244, can be used to contact an inner wall surface portion defining the shaft delivery lumen 278. For example, the distal end 234 can be pushed against the inner lumen wall portion defining the portion of the shaft delivery lumen 278 extending through the expandable distal end portion 276. Pressing the distal end 234 of the second distal portion 230 against the inner lumen wall portion can push open the expandable distal end portion 276.

In some instances, bonding together portions of the expandable distal end portion 276 can facilitate maintaining the expandable distal end portion 276 in the closed state. Pushing the distal end 234 of the second distal portion 230 against the inner lumen wall portion can break the bonding holding together portions of the expandable distal end portion 276. In some instances, the expandable distal end portion 276 can comprise a plurality of pleats. The plurality of pleats can be bonded together to maintain the expandable distal end portion 276 in the closed state. The plurality of pleats can be in a folded configuration while the expandable distal end portion 276 is in the closed state. Pressing the distal end 234 of the second distal portion 230 against the inner lumen wall portion can break the bonding between the plurality of pleats such that the plurality of pleats can assume an unfolded configuration. The plurality of pleats can unfold and assume the open state such that at least a portion of the second distal portion can be disposed through the shaft delivery lumen 278 extending through the expandable distal end portion 276. Referring to FIG. 2B, in some instances, the introducer 202 can be advanced relative to the expandable access sheath 270 such that the distal end 234 of the second distal portion 230 is disposed distally of the distal end 282 of the expandable distal end portion 276 after the introducer 202 has been deployed. At least a portion of the rigid circumferential portion 244 can be disposed distally of the distal end 282 of the expandable distal end portion 276. In some instances, the proximal end 246 of the rigid circumferential portion 244 can be disposed distally of the distal end 282 of the expandable distal end portion 276, such that the entire rigid circumferential portion 244 is distal of the sheath shaft 272. FIG. 2B shows a portion of the introducer shaft 204 can remain disposed proximally of the proximal end 286 of the sheath hub 284 after the introducer 202 has been translated distally to open the expandable distal end portion 276. In some instances, at least a portion of the distal end 264 of the introducer hub 260 can be in contact with at least a portion of the proximal end 286 of the sheath hub 284 after the introducer 202 has been deployed to open the expandable distal end portion 276.

FIGS. 2C and 2D show perspective views of the introducer 202 and the introducer 202 preloaded within the expandable access sheath 270, respectively. Referring to FIG. 2C, the externally oriented transition surface portion 238 can extend between the first externally oriented surface portion 222 defining the portion of the first distal portion 210 having the first diameter and the second externally oriented surface portion 236 defining the portion of the second distal portion 230 having the second diameter. The externally oriented transition surface portion 238 can extend from the proximal end 216 of the first distal portion 210 to a distal edge 256 of the second externally oriented surface portion 236 of the second distal portion 230. The first edge 240 of the externally oriented transition surface portion 238 can be adjacent to and in contact with a proximal edge 254 of the first externally oriented surface portion 222. The second edge 242 of the externally oriented transition surface portion 238 can be adjacent to and in contact with a distal edge 256 of the second externally oriented surface portion 236. For example, the externally oriented transition surface portion 238 can extend from the proximal edge 254 of the first externally oriented surface portion 222 to the distal edge 256 of the second externally oriented surface portion 236.

As described herein, the distal end 248 of the rigid circumferential portion 244 can be the distal end 234 of the second distal portion 230. In some instances, the externally oriented transition surface portion 238 can form at least a portion of the distal externally oriented surface 252 of the rigid circumferential portion 244. For example, the externally oriented transition surface portion 238 can be the distal externally oriented surface 252 of the rigid circumferential portion 244. The externally oriented lateral surface 250 of the rigid circumferential portion 244 can form at least a portion of the second externally oriented surface portion 236. For example, the distal externally oriented surface 252 and the externally oriented lateral surface 250 of the rigid circumferential portion 244 can be angled relative to one another, forming an angle having one or more values described herein. As described herein, the first externally oriented surface portion 222 can be parallel or substantially parallel to the longitudinal axis of the introducer shaft 204. In some instances, the externally oriented transition surface portion 238 and the externally oriented surface portion 222 can be angled relative to one another, such as forming an angle having one or more values described herein, such as the angle “Θ”. An angle formed by the distal externally oriented surface 252 and the externally oriented lateral surface 250, and/or an angle formed by the externally oriented transition surface portion 238 and the externally oriented surface portion 222, can be between about 60° and about 120°, between about 80° and about 100°, and between about 85° and about 95°, including about 90°. In some instances, the ratio of the first diameter to the second diameter, and/or the angle formed by the externally oriented transition surface portion 238 and the second externally oriented surface portion 236 and/or the externally oriented surface portion 222, can be selected such that deployment of the introducer 202 can provide consistent opening of the expandable distal end portion 276.

FIG. 2D is a perspective view of the medical access system 200 described with reference to FIG. 2A. The introducer 202 is shown preloaded within the shaft delivery lumen 278 while the expandable distal end portion 276 is in the closed state. The distal end 234 of the second distal portion 230 can be positioned at or proximate to the proximal end 280 of the expandable distal end portion 276. For example, the distal end 248 of the rigid circumferential portion 244 can be positioned at or proximate to the proximal end 280 of the expandable distal end portion 276.

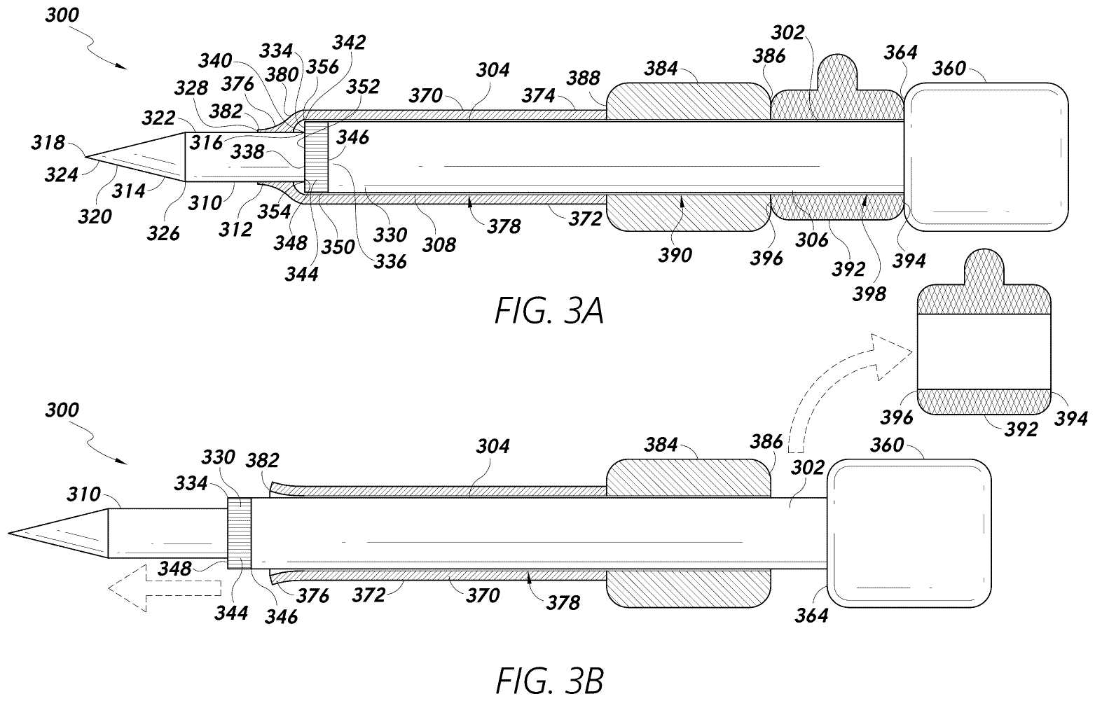

FIGS. 3A and 3B are side cross-sectional views of an example of a medical access system 300 comprising an introducer 302, an expandable access sheath 370 and a spacer member 392 configured to maintain the introducer 302 at a desired preloaded position relative to the expandable access sheath 370. FIG. 3A shows the introducer 302 preloaded within the expandable access sheath 370 and the spacer member 392 engaged with the introducer 302 and the expandable access sheath 370. FIG. 3B shows the spacer member 392 disengaged from the introducer 302 and the expandable access sheath 370 to allow translation of the introducer 302 relative to the expandable access sheath 370. The spacer member 392 can be configured to maintain the introducer 302 and the expandable access sheath 370 in the preloaded configuration, for example maintaining the relative translational position of the introducer 302 and the expandable access sheath 370. The spacer member 392 can be configured to prevent premature distal translation of the introducer 302 relative to the expandable access sheath 370.

The introducer 302 can comprise an introducer shaft 304. The introducer shaft 304 can extend distally from an introducer hub 360, including from a distal end 364 of the introducer hub 360. A proximal portion 306 of the introducer shaft 304 can be associated with an introducer hub 360, for example being coupled to the introducer hub 360, including the distal end 364 of the introducer hub 360. The expandable access sheath 370 can comprise a sheath shaft 372 extending distally from a sheath hub 384. A proximal portion 374 of the sheath shaft 372 can be associated with the sheath hub 384, for example being coupled to the sheath hub 384, including a distal end 388 of the sheath hub 384. A shaft delivery lumen 378 extending through the sheath shaft 372 can be in fluid communication with a hub delivery lumen 390 extending through the sheath hub 384. For example, while the introducer 302 is preloaded within the expandable access sheath 370, a portion of the introducer shaft 304 can be disposed through the hub delivery lumen 390 and the shaft delivery lumen 378. At least a portion of the proximal portion 306 of the introducer shaft 304 can extend proximally from a proximal end 386 of the sheath hub 384. The introducer hub 360 can be disposed proximally of the proximal end 386 of the sheath hub 384

The spacer member 392 can comprise one or more mating portions configured to engage with one or more portions of the introducer 302 and/or one or more portions of the expandable access sheath 370. In some instances, the spacer member 392 can comprise a mating portion configured to engage with a portion of the introducer shaft 304. In some instances, the spacer member 392 can comprise a mating portion configured to be positioned at least partially around a corresponding portion of the introducer shaft 304. For example, the spacer member 392 can comprise a recess 398 configured to engage with a corresponding portion of the introducer shaft 304. The recess 398 can extend through a longitudinal dimension of the spacer member 392. The recess 398 can be configured to at least partially receive the corresponding portion of the introducer shaft 304. In some instances, one or more portions of the spacer member 392 can be configured to be in contact with corresponding portions of the introducer shaft 304. In some instances, one or more portions of the spacer member 392 can be configured to be frictionally fit with corresponding portions of the introducer shaft 304.

In some instances, at least a portion of the spacer member 392 can be positioned between a proximal end 386 of the sheath hub 384 and a distal end 364 of the introducer hub 360 to maintain the relative positions of the introducer 302 and the expandable access sheath 370. In some instances, the spacer member 392 can be configured to be positioned between the sheath hub 384 and the introducer hub 360. For example, the spacer member 392 can comprise at least a portion configured to be positioned between a proximal end 386 of the sheath hub 384 and a distal end 364 of the introducer hub 360. In some instances, respective portions of the spacer member 392 can be configured to engage with one or both of the sheath hub 384 and the introducer hub 360. For example, at least a portion of a distal end 396 of the spacer member 392 can be configured to engage with the sheath hub 384. In some instances, the distal end 396 can comprise at least a portion configured to engage with at least a portion of a proximal end 386 of the sheath hub 384. In some instances, a proximal end 394 of the spacer member 392 can be configured to engage with the introducer hub 360. For example, the proximal end 394 can be configured to engage with at least a portion of the distal end 364 of the introducer hub 360.

The medical access system 300 can have one or more other features of the medical access system 200 described with reference to FIGS. 2A through 2D. For example, the introducer 302 can have a distal portion 308 comprising a first distal portion 310 having a first diameter and a second distal portion 330 having a second diameter. The first diameter can be smaller than the second diameter. In some instances, the first diameter and the second diameter can have a ratio between about 1:3 and about 2:3, including about 1:2. The first distal portion 310 can comprise a first externally oriented surface portion 322 extending along a proximal portion 312 thereof and defining the portion of the first distal portion 310 having the first diameter. A distal portion 314 of the first distal portion 310 can comprise a tapered portion 320. The tapered portion 320 can be distal of the portion having the first diameter. The tapered portion 320 can taper to a distal end 318 of the first distal portion 310. The tapered portion 320 can comprise a tapered externally oriented surface portion 324 defining a portion of the introducer shaft 304 with a decreasing diameter along a direction extending from a proximal end 316 to the distal end 318 of the first distal portion 310. The first externally oriented surface portion 322 can extend from the proximal end 316 of the first distal portion 310 to a proximal end 326 of the tapered externally oriented surface portion 324.

While the expandable distal end portion 376 is in the closed state and the introducer 302 is being advanced through the anatomical pathway, the second distal portion 330 can be configured to be disposed within the shaft delivery lumen 378. A portion of the first distal portion 310 can be disposed through a distal opening 328 at the distal end 382 of the expandable distal end portion 376. The distal end 382 of the expandable distal end portion 376 can be the distal end of the sheath shaft 372 such that at least a portion of the tapered portion 320 is positioned distally of the sheath shaft 372. In some instances, the portion of the first distal portion 310 having the first diameter can be disposed through the distal opening 328 such that the entire tapered portion 320 is disposed distally of the sheath shaft 372. The distal end 334 of the second distal portion 330 can be disposed at a position within the shaft delivery lumen 378 at the proximal end 380 of the expandable distal end portion 376. The distal end 334 of the second distal portion 330 can frictionally fit against an inner lumen wall surface portion defining the shaft delivery lumen 378 at or proximate to the proximal end 380 of the expandable distal end portion 376.

A rigid circumferential portion 344 can form at least a portion of the second distal portion 330. For example, a distal end 348 of the rigid circumferential portion 344 can be the distal end 334 of the second distal portion 330. An externally oriented lateral surface 350 of the rigid circumferential portion 344 can form at least a portion of a second externally oriented surface portion 336 defining the portion of the second distal portion 330 having the second diameter. The second distal portion 330 can comprise a proximal end 346 of the rigid circumferential portion 344. For example, the externally oriented lateral surface 350 of the rigid circumferential portion 344 can form a portion of the second externally oriented surface portion 336 defining the portion of the second distal portion 330 having the second diameter. An externally oriented transition surface portion 338 of the introducer shaft 304 can extend between the first externally oriented surface portion 322 defining the portion of the first distal portion 310 having the first diameter and the second externally oriented surface portion 336 defining the portion of the second distal portion 330 having the second diameter. For example, a first edge 340 of the externally oriented transition surface portion 338 can be adjacent to and in contact with the first externally oriented surface portion 322, such as a proximal edge 354 of the first externally oriented surface portion 322. A second edge 342 of the externally oriented transition surface portion 338 can be adjacent to and in contact with the second externally oriented surface portion 336, such as a distal edge 356 of the second externally oriented surface portion 336. The externally oriented transition surface portion 338 can be a distal externally oriented surface 352 of the rigid circumferential portion 344. In some instances, the externally oriented transition surface portion 338 and the second externally oriented surface portion 336 can form an angle between about 60° and about 120°, including about 80° and about 100°. In some instances, the angle can be about 85° and about 95°. In some instances, the externally oriented transition surface portion 338 is perpendicular or substantially perpendicular to the second externally oriented surface portion 336.

Referring to FIG. 3B, the spacer member 392 can be removed to allow distal advancement of the introducer 302 relative to the expandable access sheath 370. For example, after removal of the spacer member 392 from around the introducer shaft 304, and/or any engagement between the spacer member 392 and the introducer hub 360 and/or sheath hub 384, the introducer shaft 304 can be advanced within the shaft delivery lumen of the expandable access sheath 370. The distal end 334 of the second distal portion 330 of the introducer shaft 304 can be pushed against the expandable distal end portion 376 of the sheath shaft 372 to cause the expandable distal end portion 376 to transform from the closed state to an open state. The introducer 302 can be advanced until the second distal portion 330 is disposed distally of a distal end 382 of the sheath shaft 372. In some instances, the introducer 302 can be advanced until the distal end 364 of the introducer hub 360 is in contact with the proximal end 386 of the sheath hub 384.

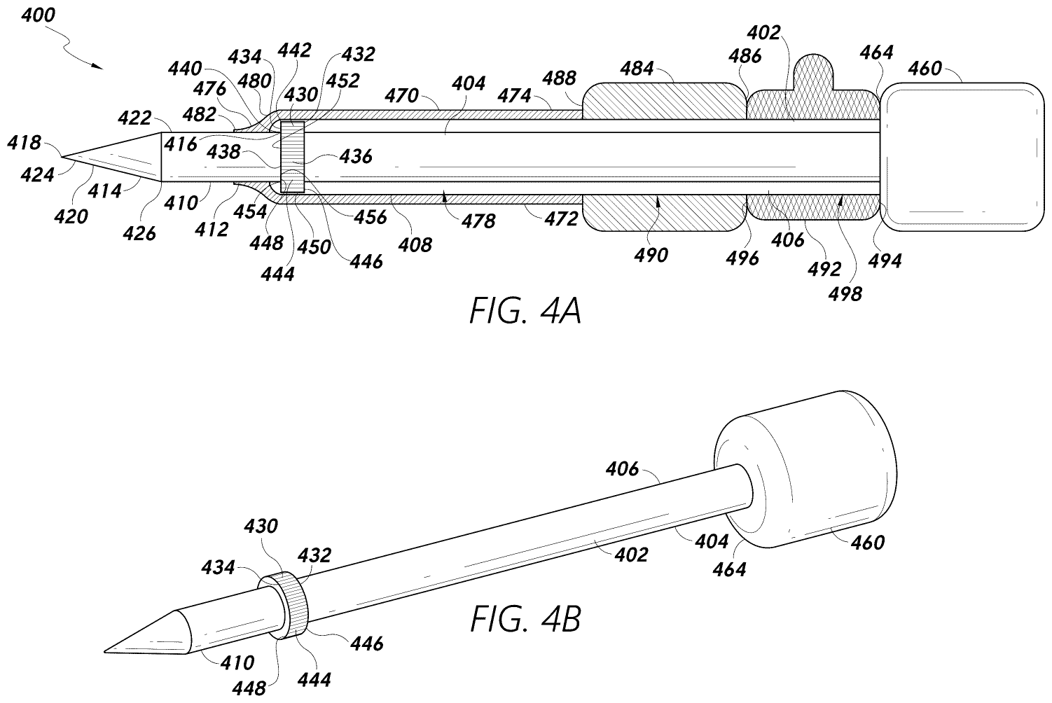

FIG. 4A is a cross-sectional view, and FIG. 4B is a perspective view, of an example of a medical access system 400 that includes an expandable access sheath 470 and introducer 402 with an introducer shaft 404 configured to be disposed partially within the expandable access sheath 470, where the introducer shaft 404 can comprise a second distal portion 430 having a second diameter and a portion proximal of the second distal portion 430 having a third diameter. The third diameter can be smaller than the second diameter. In some instances, a rigid circumferential portion 444 can form at least a portion of the second distal portion 430. In some instances, the second distal portion 430 can be the rigid circumferential portion 444. For example, a distal end 448 of the rigid circumferential portion 444 can be a distal end 434 of the second distal portion 430. A proximal end 446 of the rigid circumferential portion 444 can be a proximal end 432 of the second distal portion 430. A second externally oriented surface portion 436 can define the portion of the second distal portion 430 having the second diameter. An externally oriented lateral surface 450 of the rigid circumferential portion 444 can form at least a portion of the second externally oriented surface portion 436. Portions of the introducer shaft 404 proximal of the proximal end 446 of the rigid circumferential portion 444 can have the third diameter. A proximal portion 406 of the introducer shaft 404 can have the third diameter. For example, portions of the introducer shaft 404 proximal of the proximal end 446 of the rigid circumferential portion 444 can have a smaller diameter than that of the second distal portion 430. In some instances, portions proximal of the proximal end 446 of the rigid circumferential portion 444 comprising a smaller diameter can facilitate navigation of the introducer shaft 404 through anatomical pathways. For example, a size, such as a diameter, of the rigid circumferential portion 444 can be selected to provide desired engagement with an inner lumen wall portions of the expandable access sheath 470, while portions of the introducer shaft 404 proximal of the proximal end 446 of the rigid circumferential portion 444 can have a reduced profile to facilitate navigation through tortuous anatomical pathways.

The expandable access sheath 470 can comprise a sheath shaft 472 with a shaft delivery lumen 478 extending therethrough. The sheath shaft 472 can comprise a proximal portion 474 associated with a sheath hub 484 and an expandable distal end portion 476. While the expandable distal end portion 476 is in a closed state, at least a portion of the introducer shaft 404 can be configured to be disposed within the shaft delivery lumen 478 of the sheath shaft 472. A distal end 434 of the second distal portion 430 can be configured to engage with one or more portions of the inner lumen wall portion defining the shaft delivery lumen 478. The distal end 448 of the rigid circumferential portion 444 can be configured to engage with one or more portions of the inner lumen wall portion defining the shaft delivery lumen 478. A size, such as a diameter, of the rigid circumferential portion 444 can be selected to provide desired engagement with inner lumen wall portions of the sheath shaft 472. In some instances, while preloaded within the shaft delivery lumen 478, the distal end 448 of the rigid circumferential portion 444 can be configured to be positioned within the shaft delivery lumen 478 at a proximal end 480 of the expandable distal end portion 476. In some instances, the distal end 434 of the second distal portion 430, such as the distal end 448 of the rigid circumferential portion 444, can frictionally fit against an inner lumen wall surface portion defining the shaft delivery lumen 478 at or proximate to the proximal end 480 of the expandable distal end portion 476.

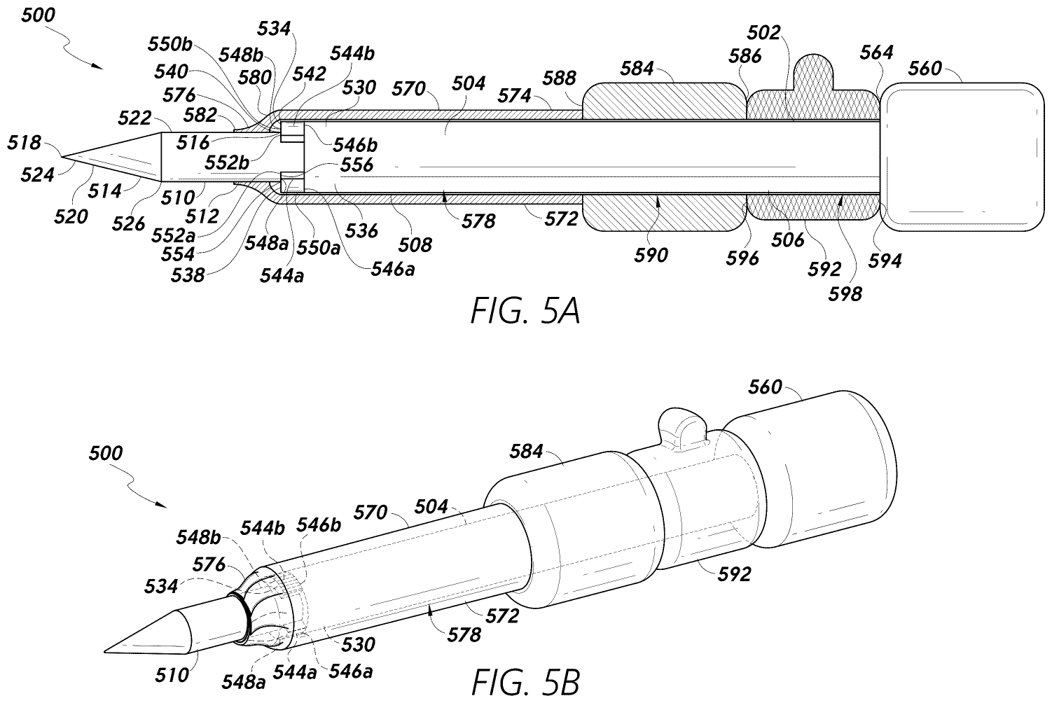

FIG. 5A is a side cross-sectional view, and FIG. 5B is a perspective view, of another example of a medical access system 500. The medical access system 500 can include an expandable access sheath 570 and introducer 502 with an introducer shaft 504 configured to be disposed partially within the expandable access sheath 570. The sheath shaft 504 can comprise a first distal portion 510 having a first diameter and that is distal of a second distal portion 530. The second distal portion 530 of the introducer shaft 504 can comprise at least a portion having a second diameter. The second distal portion 530 can have a distal end 534 disposed around only a portion of a circumference of the introducer shaft 504. The second distal portion 530 can comprise a rigid portion that is disposed partially around the circumference of the introducer shaft 504. For example, the second distal portion 530 can comprise a first and second partial circumferential portion 544a, 544b spaced from one another. The first and second partial circumferential portions 544a, 544b can be distinct rigid portions each disposed around a respective portion of the circumference of the introducer shaft 504.

In some instances, the first and second partial circumferential portions 544a, 544b can form respective portions of the second distal portion 530. For example, a distal end 548a, 548b of the first and second partial circumferential portion 544a, 544b can form respective portions of the distal end 534 of the second distal portion 530. The second distal portion 530 can comprise proximal ends 546a, 546b of the first and second partial circumferential portions 544a, 544b. An externally oriented transition surface portion 538 can extend between a first externally oriented surface portion 522 defining the portion of the first distal portion 510 having the first diameter and a second externally oriented surface portion 536 defining the portion of the second distal portion 530 having the second diameter. A first edge 540 of the externally oriented transition surface portion 538 can be adjacent to and in contact with a proximal edge 554 of the first externally oriented surface portion 522. A second edge 542 of the externally oriented transition surface portion 538 can be adjacent to and in contact with a distal edge 556 of the second externally oriented surface portion 536. Distally oriented surfaces 552a, 552b of the rigid partial circumferential portions 544a, 544b can form at least a portion of the externally oriented transition surface portion 538. The externally oriented lateral surfaces 550a, 550b of the rigid partial circumferential portions 544a, 544b can form at least a portion of the second externally oriented surface portion 536.

The expandable access sheath 570 can comprise a sheath shaft 572 having a proximal portion 574 associated with a sheath hub 584 and an expandable distal end portion 576. A shaft delivery lumen 578 can extend through the sheath shaft 572. While the expandable distal end portion 576 is in a closed state, a portion of the introducer shaft 504 can be configured to be disposed within the shaft delivery lumen 578 such that a distal end 534 of the second distal portion 530 can be configured to engage with one or more portions of the inner lumen wall portion defining the shaft delivery lumen 578. For example, the distal ends 548a, 548b of the rigid circumferential portions 544a, 544b can be configured to engage with one or more portions of the inner lumen wall portion defining the shaft delivery lumen 578. A configuration of the rigid partial circumferential portions 544a, 544b can be selected to provide desired engagement with inner lumen wall portions of the sheath shaft 572, including a frictional fit with the inner lumen wall portions. In some instances, while preloaded within the shaft delivery lumen 578, the distal ends 548a, 548b of the rigid circumferential portions 544a, 544b can be configured to be positioned within the shaft delivery lumen 578 at or proximate to a proximal end 580 of the expandable distal end portion 576. In some instances, distal ends 548a, 548b of the rigid circumferential portions 544a, 544b can frictionally fit against an inner lumen wall surface portion defining the shaft delivery lumen 578 at or proximate to the proximal end 580 of the expandable distal end portion 576.

The medical access system 400 described with reference to FIGS. 4A and 4B and the medical access system 500 described with reference to FIGS. 5A and 5B can have one or more other features of the medical access system 300 described with reference to FIGS. 3A and 3B. For example, the sheath shaft 472, 572 can be coupled to the sheath hub 484, 584 including a distal end 488, 588 of the sheath hub 484, 584. The shaft delivery lumen 478, 578 extending through the sheath shaft 472, 572 can be in fluid communication with a hub delivery lumen 490, 590 extending through the sheath hub 484, 584. A distal portion 408, 508 of each of the introducer shafts 404, 504 can comprise a first distal portion 410, 510 having a first diameter and being distal of the second distal portion 430, 530. The first diameter can be smaller than the second diameter. In some instances, the first diameter and the second diameter can have a ratio between about 1:3 and about 2:3, including about 1:2. A first externally oriented surface portion 422, 522 extending along a proximal portion 412, 512 of the first distal portion 410, 510 can define the portion of the first distal portion 410, 510 having the first diameter. A distal portion 414, 514 of the first distal portion 410, 510 can comprise a tapered portion 420, 520. The tapered portion 420, 520 can be distal of the portion having the first diameter. The tapered portion 420, 520 can taper to a distal end 418, 518 of the first distal portion 410, 510. The tapered portion 420, 520 can comprise a tapered externally oriented surface portion 424, 524 defining a portion of the introducer shaft 404, 504 with a decreasing diameter extending along a direction from a proximal end 416, 516 to the distal end 418, 518 of the first distal portion 410, 510. The first externally oriented surface portions 422, 522 can extend from proximal ends 416, 516 of the first distal portions 410, 510 to proximal ends 426, 526 of the tapered externally oriented surface portion 424, 524. While the expandable distal end portion 476, 576 is in the closed state and the introducer 402, 502 is being advanced through the anatomical pathway, a portion of the first distal portion 410, 510, such as the portion having the first diameter, can be disposed through a distal opening at the distal end 482, 582 of the expandable distal end portion 476, 576. The distal end 482, 582 of the expandable distal end portion 476, 576 can be the distal end of the sheath shaft 472, 572, such that at least a portion of the tapered portion 420, 520 is positioned distally of the sheath shaft 472, 572. For example, at least a portion of the tapered portion 420, 520 can be disposed distally of the sheath shaft 472, 572 to facilitate navigation of the medical access system 400 through tortuous anatomical pathways.

An externally oriented transition surface portion 438, 538 of the introducer shaft 404, 504 extending between the first externally oriented surface portion 422, 522 and the second externally oriented surface portion 436, 536, and the second externally oriented surface portion 436, 536, can form an angle between about 60° and about 120°, including between about 80° and about 100°, and about 85° and about 95°, including about 90°. A first edge 440, 540 of the externally oriented transition surface portion 438, 538 can be adjacent to and in contact with the first externally oriented surface portion 422, 522, such as a proximal edge 454, 554 of the first externally oriented surface portion 422, 522. A second edge 442, 542 of the externally oriented transition surface portion 438, 538 can be adjacent to and in contact with the second externally oriented surface portion 436, 536, such as a distal edge 456, 556 of the second externally oriented surface portion 436, 536. The externally oriented transition surface portion 438, 538 can be a distal externally oriented surface 452, 552a, 552b of the rigid circumferential portion 444, 544a, 544b.

In some instances, the medical access systems 400, 500 can each comprise a spacer member 492, 592 to maintain a relative position between the expandable access sheath 470, 570 and the introducer 402, 502 while the introducer 402, 502 is preloaded within the expandable access sheath 470, 570. For example, each spacer member 492, 592 can comprise a recess 498, 598 extending through a longitudinal dimension of the spacer member 492, 592 that is configured to engage with a corresponding portion of the introducer shaft 404, 504 extending proximally of a proximal end 486, 586 of a sheath hub 484, 584. The recess 498, 598 can engage with the proximal portion 406, 506 of the introducer shaft 404, 504. In some instances, the recesses 498, 598 can extend from a proximal end 494, 594 to a distal end 496, 596 of the spacer member 492, 592. In some instances, at least a portion of a distal end 496, 596 of the spacer member 492, 592 can be configured to engage with the sheath hub 484, 584, such as at least a portion of a proximal end 486, 586 of the sheath hub 484, 584. In some instances, at least a portion of a proximal end 494, 594 of the spacer member 492, 592 can be configured to engage with at least a portion of the distal end 464, 564 of the introducer hub 460, 560.

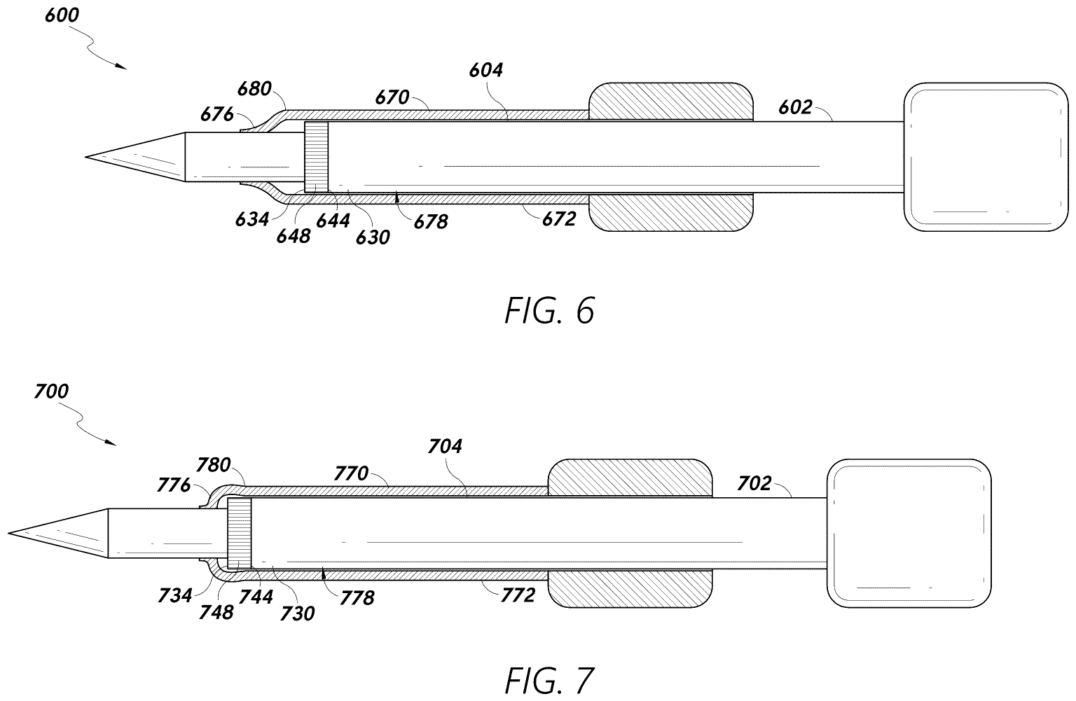

FIG. 6 and FIG. 7 are side cross-sectional views of different examples of medical access systems 600, 700 having an introducer 602, 702 preloaded within a respective expandable access sheath 670, 770. Each of the introducers 602, 702 can comprise an introducer shaft 604, 704 configured to be disposed partially within a shaft delivery lumen 678, 778 of a sheath shaft 672, 772 of the expandable access sheath 670, 770. Each sheath shaft 672, 772 can comprise an expandable distal end portion 676, 776 configured to assume a closed state or an open state. FIGS. 6 and 7 show the introducers 602, 702 preloaded within the expandable access sheaths 670, 770 while the expandable distal end portions 676, 776 are in the closed state. FIG. 6 shows a distal end 634 of a second distal portion 630 of the introducer shaft 604 disposed within the shaft delivery lumen 678 at a position proximal of the expandable distal end portion 676. FIG. 7 shows a distal end 734 of a second distal end portion 730 of the introducer shaft 704 disposed within the shaft delivery lumen 778 at a position within the expandable distal end portion 776.

Each of the second distal portions 630, 730 of the introducer shafts 604, 704 can comprise a rigid circumferential portion 644, 744 that forms at least a portion of the respective second distal portion 630, 730. For example, a distal end 648, 748 of each of the rigid circumferential portions 644, 744 can be the distal end 634, 734 of the respective second distal portion 630, 730. Referring to FIG. 6, the introducer shaft 604 can be positioned within the shaft delivery lumen 678 such that the distal end 648 of the rigid circumferential portion 644 can be proximal of the proximal end 680 of the expandable distal end portion 676. Referring to FIG. 7, the introducer shaft 704 can be positioned within the shaft delivery lumen 778 such that the distal end 748 of the rigid circumferential portion 744 can be distal of the proximal end 780 of the expandable distal end portion 776. The distal end 748 of the rigid circumferential portion 744 can be disposed within the shaft delivery lumen 778 at a position within the expandable distal end portion 776.

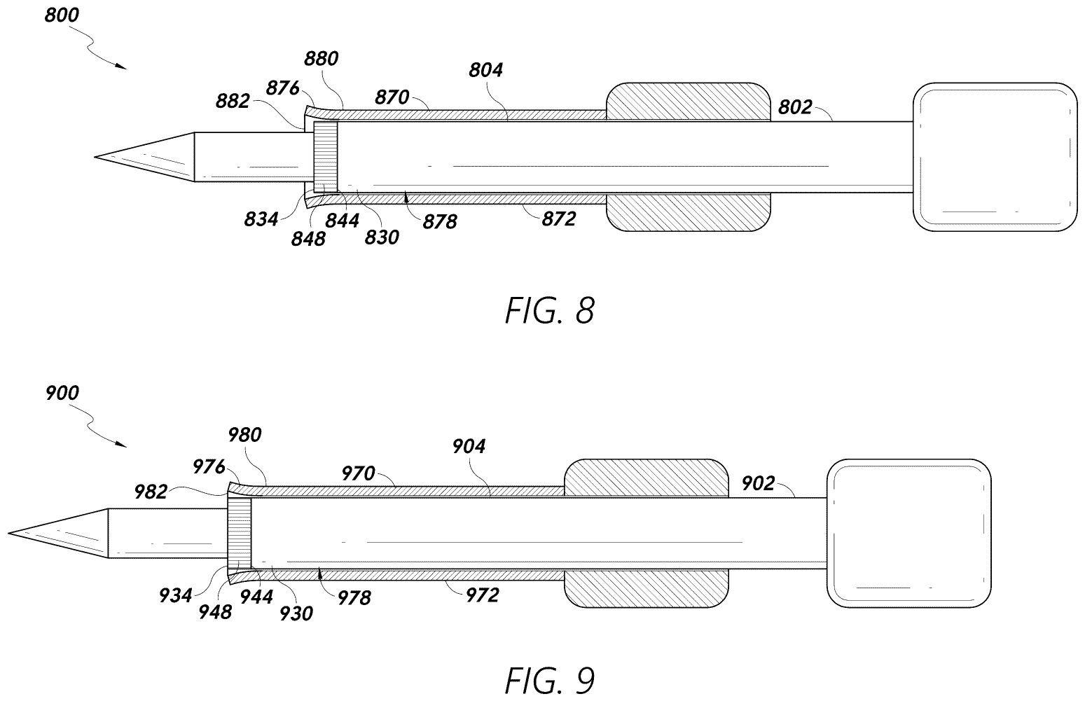

FIGS. 8 and 9 are side cross-sectional views of examples of medical access systems 800, 900 comprising introducers 802, 902 disposed partially within a respective expandable access sheath 870, 970, where the introducers 802, 902 are in a deployed state. Each expandable access sheath 870, 970 can comprise a sheath shaft 872, 972 having an expandable distal end portion 876, 976. Each of the introducers 802, 902 can comprise an introducer shaft 804, 904 configured to be disposed partially within a respective shaft delivery lumen 878, 978 of the sheath shafts 872, 972. The introducers 802, 902 have been translated distally relative to the respective expandable access sheath 870,970 to transform the expandable distal end portions 876, 976 from a closed state to an open state.

FIG. 8 shows a distal end 834 of a second distal portion 830 of the introducer shaft 804 disposed within the shaft delivery lumen 878 at a position proximal of a distal end 882 of the expandable distal end portion 876. FIG. 9 shows a distal end 934 of a second distal end portion 930 of the introducer shaft 904 disposed at a distal end 982 of the expandable distal end portion 976. The expandable distal end portions 876, 976 are shown in an open state. The introducers 802, 902 have been advanced distally relative to the expandable access sheaths 870, 970 such that the distal ends 834, 934 of the second distal portions 830, 930 have been pushed against respective inner lumen wall portions of the expandable distal end portions 876, 976 to cause the expandable distal end portions 876, 976 to transform from a closed state to the open state. In some instances, a rigid circumferential portion 844, 944 can form a portion of the respective second distal portion 830, 930 such that a distal end 848, 948 of the rigid circumferential portion is the distal end 834, 934 of the second distal portion 830, 930. For example, in FIG. 8, the distal end 848 of the rigid circumferential portion 844 can be proximal of the distal end 882 of the expandable distal end portion 876. In FIG. 9, the distal end 948 of the rigid circumferential portion 944 can be at or proximate to the distal end 982 of the expandable distal end portion 976

In some instances, while the introducer is in the deployed state, distal ends 834, 934 of the second distal portions 830, 930 can be configured to remain within the shaft delivery lumen 878, 978 to reduce or prevent injury to surrounding tissue. In some instances, tissue damage may result from contact between the distal ends 834, 934 of the second distal portions 830, 930 and tissue of the lumen, vessel, and/or chamber into which the medical access systems 800, 900 is positioned. The introducers 802, 902 can be translated distally relative to the expandable access sheaths 870, 970 to transform the expandable distal end portions 876, 976 from the closed state to the open state while maintaining the second distal portions 830, 930 within the shaft delivery lumens 878, 978.

As described herein, an introducer can be preloaded within a shaft delivery lumen at a position proximal of, at, or distal of, a proximal end of an expandable distal end portion. For example, Referring to FIG. 8, advancing the introducer 802 relative to the sheath shaft 872 to open the expandable distal end portions 876 can comprise advancing the distal end 834 of the second distal portions 830 through a portion of the shaft delivery lumen 878 proximal of the proximal end 880 of the expandable distal end portion 876 and through a portion of the shaft delivery lumen 878 extending through the expandable distal end portion 876. In some instances, advancing the introducer 802 can comprise advancing the distal end 834 of the second distal portions 830 only through a portion of the shaft delivery lumen 878 extending through the expandable distal end portion 876. Referring to FIG. 9, in some instances, advancing the introducer 902 can comprise advancing the distal end 934 of the second distal portions 930 through a portion of the shaft delivery lumen 978 proximal of the proximal end 980 of the expandable distal end portion 976 and through an entire or substantially an entire length of the shaft delivery lumen 978 extending through the expandable distal end portion 976. In some instances, advancing the introducer 902 can comprise advancing the distal end 934 of the second distal portions 930 only through the entire or substantially entire length of the shaft delivery lumen 978 extending through the expandable distal end portion 976.

It will be understood that the medical access systems 600, 700, 800, 900 described with reference to FIGS. 6, 7, 8 and 9 can comprise one or more other features of one or more of the other medical access systems described herein.

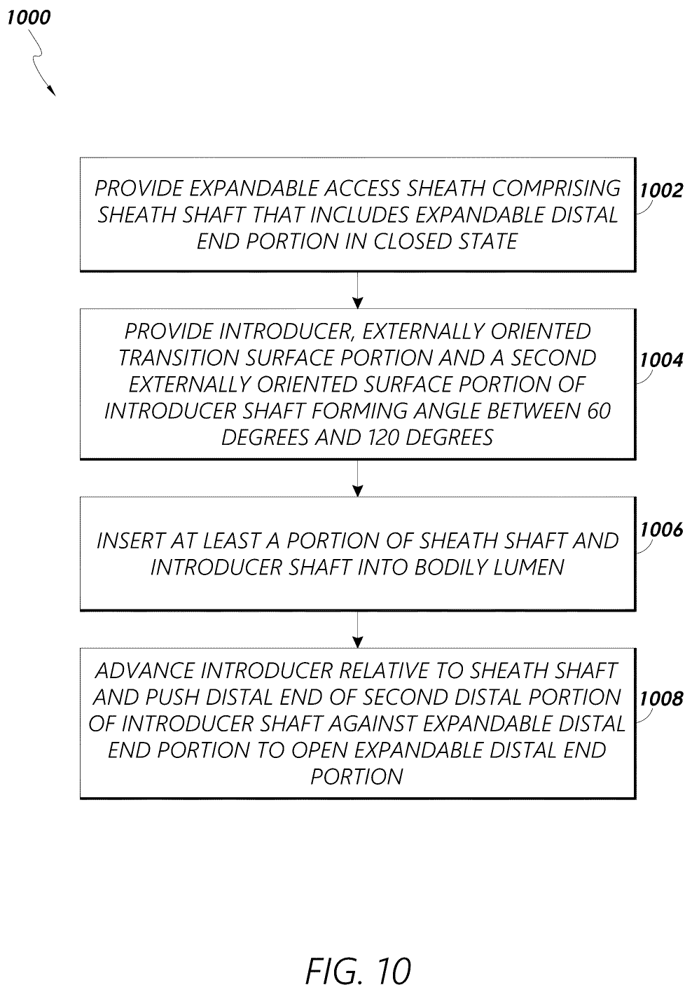

FIG. 10 is a process flow diagram of an example of a delivery process 1000 for delivering a medical device using a medical access system having one or more features described herein. In block 1002, the process can involve providing an expandable access sheath. The expandable access sheath can comprise features of one or more expandable access sheaths described herein. The expandable access sheath can comprise a sheath shaft that includes an expandable distal end portion. The expandable distal end portion can be provided in a closed state. A shaft delivery lumen can extend through the sheath shaft.

In block 1004, the process can involve providing an introducer comprising an introducer shaft disposed at least partially within the shaft delivery lumen of the sheath shaft. The introducer shaft can comprise an externally oriented transition surface portion extending between a first distal portion and a second distal portion. The second distal portion can comprise a diameter larger than that of the first distal portion. The second distal portion can be proximal of the first distal portion. The externally oriented transition surface portion and a second externally oriented surface portion of the second distal portion can form an angle between about 60° and about 120°.

In block 1006, the process can involve inserting at least a portion of the sheath shaft and the introducer shaft into a bodily lumen. In block 1008, the process can involve advancing the introducer relative to the sheath shaft. A distal end of the second distal portion of the introducer shaft can be pushed against an inner wall surface portion of the sheath shaft defining the shaft delivery lumen extending through the expandable distal end portion to thereby cause the expandable distal end portion to transform to an open state.

In some instances, pushing the distal end of the second distal portion against the inner wall surface portion can comprise pushing the distal end against a proximal portion of the inner wall surface portion defining the shaft delivery lumen extending through the expandable distal end portion. In some instances, pushing the distal end of the second distal portion against the inner wall surface portion can comprise pushing the distal end against a proximal end of the inner wall surface portion defining the shaft delivery lumen extending through the expandable distal end portion.

As described herein, the expandable distal end portion can comprise a plurality of pleats. The plurality of pleats can be in a folded configuration while the expandable distal end portion is in the closed state. In some instances, pushing the distal end of the second distal portion against the expandable distal end portion can comprise pushing the distal end against a plurality of pleats of the expandable distal end portion to cause unfolding of the plurality of pleats for transforming the expandable distal end portion to the open state. The plurality of pleats can be bonded together to facilitate maintaining the plurality of pleats in the folded configuration. In some instances, pushing the distal end against the plurality of pleats can comprise pushing the distal end against the plurality of pleats to cause breaking of bonding used to maintain the plurality of pleats in a folded configuration. Breaking of the bonding between the plurality of pleats can allow unfolding of the plurality of pleats. For example, the introducer can be advanced relative to the sheath shaft such that the distal end of the second distal portion can be advanced through the shaft delivery lumen extending through the expandable distal end portion to cause the plurality of pleats to unfold.

While the introducer is preloaded within the expandable access sheath, the distal end of the second distal portion can be positioned proximal of a proximal end of the expandable distal end portion. In some instances, advancing the introducer relative to the sheath shaft can comprise advancing the distal end of the second distal portion through both a portion of the shaft delivery lumen proximal of the expandable distal end portion and at least a portion of the shaft delivery lumen extending through the expandable distal end portion. In some instances, advancing the introducer relative to the sheath shaft can comprise advancing the distal end of the second distal portion to a position distal of a distal end of the sheath shaft. In some instances, advancing the introducer relative to the sheath shaft can comprise advancing the distal end of the second distal portion to a position proximal of a distal end of the sheath shaft. In some instances, advancing the introducer relative to the sheath shaft can comprise advancing the distal end of the second distal portion to a position at a distal end of the sheath shaft. In some instances, advancing the introducer relative to the sheath shaft can comprise advancing the distal end of the second distal portion through an entire length of the shaft delivery lumen extending through expandable distal end portion.

In some instances, while the introducer is preloaded within the expandable access sheath, the distal end of the second distal portion can be positioned at a proximal end of the expandable distal end portion. In some instances, while the introducer is preloaded within the expandable access sheath, the distal end of the second distal portion can be positioned at a position within the expandable distal end portion. For example, providing the introducer can comprise providing a distal end of the second distal portion of the introducer shaft frictionally fitted against a respective inner wall surface portion defining the shaft delivery lumen extending through the expandable distal end portion.

In some instances, the distal end of the second distal portion can comprise a circumferential portion. Providing the distal end of the second distal portion of the introducer shaft frictionally fitted against a respective inner wall surface portion can comprise providing the distal end of the second distal end portion frictionally fitted against a respective circumferential inner wall surface portion defining the shaft delivery lumen.

The medical access system can comprise a spacer member configured to maintain a position of the introducer relative to the expandable access sheath, such as while the introducer is preloaded within the expandable access sheath. The spacer member can be engaged with the expandable access sheath and/or introducer while the expandable access sheath and introducer are advanced to a target position. The introducer can be advanced relative to the expandable access sheath for transforming the expandable distal end portion of the expandable access sheath to the open state. In some instances, a process for delivering a medical device to a target site can comprise removing the spacer member from between a proximal end of a sheath hub and a distal end of an introducer hub prior to advancing the introducer relative to the shaft. In some instances, removing the spacer member can comprise removing the spacer member from around the introducer shaft. In some instances, removing the spacer member can comprise sliding a corresponding portion of the introducer shaft out from a recess extending through a longitudinal dimension of the spacer member. In some instances, removing the spacer member can comprise disengaging at least a portion of a distal end of the spacer member from at least a portion of a proximal end of a sheath hub. In some instances, removing the spacer member can comprise disengaging at least a portion of a proximal end of the spacer member from at least a portion of a distal end of an introducer hub.

A medical device can be advanced through the shaft delivery lumen after the expandable distal end portion has been opened. In some instances, the introducer can be retracted relative to the sheath shaft to remove the introducer. Subsequently, the medical device can be advanced through the shaft delivery lumen. For example, at least a portion of a delivery catheter carrying the medical device can be advanced through the shaft delivery lumen while the expandable distal end portion is in the open state. As described herein, medical access systems can be used to treat various conditions of the heart. In some instances, the delivery catheter carrying the medical device can be advanced to the heart. For example, a distal portion of the delivery catheter can be advanced to the heart or to a target position proximate to the heart. The medical device can then be deployed from the delivery catheter.

In some instances, inserting the at least a portion of the sheath shaft and introducer shaft into the bodily lumen can comprise inserting the at least a portion of the sheath shaft and introducer shaft into a femoral artery. For example, the sheath shaft carrying the introducer shaft can be inserted into an access opening formed on a right femoral artery or a left femoral artery. In some instances, the expandable access sheath can be disposed within the right or left femoral artery. A medical device, including a delivery catheter carrying the medical device, can be advanced through the sheath shaft disposed within the femoral artery. For example, the delivery catheter carrying the medical device can be advanced through the shaft delivery lumen, and from the femoral artery into the aorta. The delivery catheter carrying the medical device can subsequently be advanced through the aorta to the heart. In some instances, inserting the at least a portion of the sheath shaft and introducer shaft into the bodily lumen can comprise inserting the at least a portion of the sheath shaft and introducer shaft into another arterial access opening, or a venous access opening, including a femoral vein, internal jugular vein, or subclavian vein.

### Additional Description of Examples

Provided below is a list of examples, each of which may include aspects of any of the other examples disclosed herein. Furthermore, aspects of any example described above may be implemented in any of the numbered examples provided below.

Example 1: A medical access system comprising an expandable access sheath having a sheath hub, and a sheath shaft extending distally from the hub and comprising an expandable distal end portion. The sheath shaft can comprise a shaft delivery lumen extending therethrough. The system can include an introducer comprising an introducer shaft, the introducer shaft being configured to be at least partially disposed within the shaft delivery lumen while the expandable distal end portion is in a closed state. The introducer shaft can comprise a first distal portion having a first diameter, and a second distal portion proximal of the first distal portion, the second distal portion comprising a second diameter larger than the first diameter. The introducer can have an externally oriented transition surface portion extending between a first externally oriented surface portion defining a portion of the first distal portion having the first diameter and a second externally oriented surface portion defining a portion of the second distal portion having the second diameter, the externally oriented transition surface portion and the second externally oriented surface portion forming an angle between about 80 degrees and about 100 degrees.

Example 2: The system of any example herein, in particular example 1, wherein the angle is between 85 degrees and 95 degrees.

Example 3: The system of any example herein, in particular example 1 or 2, wherein the externally oriented transition surface portion is perpendicular to the second externally oriented surface.

Example 4: The system of any example herein, in particular examples 1 to 3, wherein the second externally oriented surface is parallel to a longitudinal axis of the introducer.

Example 5: The system of any example herein, in particular examples 1 to 4, wherein the first externally oriented surface is parallel to a longitudinal axis of the introducer.

Example 6: The system of any example herein, in particular examples 1 to 5, wherein the first diameter and the second diameter have a ratio between 1:3 and 2:3.

Example 7: The system of any example herein, in particular example 6, wherein the first diameter and the second diameter have a ratio of 1:2.

Example 8: The system of any example herein, in particular examples 1 to 7, wherein the introducer shaft further comprises a proximal portion proximal of the second distal portion, the proximal portion having a third diameter smaller than the second diameter.

Example 9: The system of any example herein, in particular examples 1 to 7, wherein a remaining portion of the introducer shaft proximal of the second distal portion comprises the second diameter.

Example 10: The system of any example herein, in particular examples 1 to 9, wherein, while the expandable distal end portion is in the closed state, a distal end of the second distal portion is configured to be positioned within the shaft delivery lumen at a position proximal of a proximal end of an inner wall surface portion defining the shaft delivery lumen extending through the expandable distal end portion.

Example 11: The system of any example herein, in particular examples 1 to 9, wherein a distal end of the second distal portion frictionally fits against a respective inner wall surface portion defining the shaft delivery lumen extending through the expandable distal end portion.

Example 12: The system of any example herein, in particular example 11, wherein the distal end of the second distal portion comprises a circumferential portion and is configured to frictionally fit against a respective circumferential inner wall surface portion defining the shaft delivery lumen.

Example 13: The system of any example herein, in particular example 11 or 12, wherein, while the expandable distal end portion is in the closed state, the distal end of the second distal portion is configured to be positioned against a proximal portion of the inner wall surface portion defining the shaft delivery lumen extending through the expandable distal end portion.

Example 14: The system of any example herein, in particular example 11 or 12, wherein, while the expandable distal end portion is in the closed state, the distal end of the second distal portion is configured to be positioned against a proximal end of the inner wall surface portion defining the shaft delivery lumen extending through the expandable distal end portion.

Example 15: The system of any example herein, in particular examples 1 to 14, wherein the distal end of the second distal portion extends at least partially around a circumference of the introducer shaft.

Example 16: The system of any example herein, in particular example 15, wherein the distal end of the second distal portion is a circumferential portion extending circumferentially around the introducer shaft.

Example 17: The system of any example herein, in particular example 15 or 16, wherein the second distal portion is a rigid circumferential portion.

Example 18: The system of any example herein, in particular example 17, wherein the rigid circumferential portion is a metallic ring.

Example 19: The system of any one of any example herein, in particular examples 1 to 18, further comprising a spacer member configured to prevent distal translation of the introducer relative to the sheath.

Example 20: The system of any example herein, in particular example 19, wherein the introducer further comprises an introducer hub, the introducer shaft extending distally from the introducer hub, and the spacer member comprises at least a portion being configured to be positioned between a proximal end of the sheath hub and a distal end of the introducer hub.

Example 21: The system of any example herein, in particular example 20, wherein the spacer member is between the proximal end of the sheath hub and the distal end of the introducer hub.

Example 22: The system of any example herein, in particular examples 19 to 21, wherein the spacer member comprises a mating portion configured to be positioned at least partially around a corresponding portion of the introducer shaft.

Example 23: The system of any example herein, in particular example 22, wherein the mating portion of the spacer member comprises a recess extending through a longitudinal dimension, the recess being configured to at least partially receive the corresponding portion of the introducer shaft.

Example 24: The system of any example herein, in particular examples 1 to 23, wherein the expandable distal end portion comprises a plurality of pleats, the pleats being in a folded configuration while the expandable distal end portion is in the closed state and in an unfolded configuration while the expandable distal end portion is in an open state.

Example 25: The system of any example herein, in particular example 24, wherein the plurality of pleats are bonded to maintain the expandable distal end portion in the closed state.

Example 26: The system of any example herein, in particular examples 1 to 25, wherein the first distal portion comprises a tapered portion tapering to a distal end of the introducer shaft.

Example 27: The system of any example herein, in particular examples 1 to 26, wherein the expandable access sheath and the introducer are sterilized.

Example 28: A method of delivering a medical device, the method comprising providing an expandable access sheath comprising a sheath shaft that includes an expandable distal end portion in a closed state, the sheath shaft comprising a shaft delivery lumen extending therethrough. The method can include providing an introducer comprising an introducer shaft at least partially disposed within the shaft delivery lumen, the introducer shaft comprising an externally oriented transition surface portion extending between a first distal portion and a second distal portion, the second distal portion comprising a diameter larger than that of the first distal portion and being proximal of the first distal portion, the externally oriented transition surface portion and a second externally oriented surface portion of the second distal portion angled relative to one another, such as forming an angle between about 60 degrees and about 120 degrees. The method can include inserting at least a portion of the sheath shaft and the introducer shaft into a bodily lumen, and advancing the introducer relative to the sheath shaft and pushing a distal end of the second distal portion of the introducer shaft against an inner wall surface portion defining the shaft delivery lumen extending through the expandable distal end portion to thereby cause the expandable distal end portion to transform to an open state.

Example 29: The method of any example herein, in particular example 28, wherein pushing the distal end of the second distal portion against the inner wall surface portion comprises pushing the distal end against a proximal portion of the inner wall surface portion defining the shaft delivery lumen extending through the expandable distal end portion.

Example 30: The method of any example herein, in particular example 28, wherein pushing the distal end of the second distal portion against the inner wall surface portion comprises pushing the distal end against a proximal end of the inner wall surface portion defining the shaft delivery lumen extending through the expandable distal end portion.

Example 31: The method of any example herein, in particular examples 28 to 30, wherein pushing the distal end of the second distal portion against the expandable distal end portion comprises pushing the distal end against a plurality of pleats of the expandable distal end portion to cause unfolding of the plurality of pleats for transforming the expandable distal end portion to the open state.

Example 32: The method of any example herein, in particular example 31, wherein pushing the distal end against the plurality of pleats comprises pushing the distal end against the plurality of pleats to cause breaking of bonding maintaining the plurality of pleats in a folded configuration and unfolding of the plurality of pleats.

Example 33: The method of any example herein, in particular examples 28 to 32, wherein advancing the introducer relative to the sheath shaft comprises advancing the distal end of the second distal portion through a portion of the shaft delivery lumen proximal of the expandable distal end portion.

Example 34: The method of any example herein, in particular examples 28 to 33, wherein advancing the introducer relative to the sheath shaft comprises advancing the distal end of the second distal portion to a position distal of a distal end of the sheath shaft.

Example 35: The method of any example herein, in particular examples 28 to 33, wherein advancing the introducer relative to the sheath shaft comprises advancing the distal end of the second distal portion to a position at a distal end of the sheath shaft.

Example 36: The method of any example herein, in particular example 34 or 35, wherein advancing the introducer relative to the sheath shaft comprises advancing the distal end of the second distal portion through an entire length of the shaft delivery lumen extending through the expandable distal end portion.

Example 37: The method of any example herein, in particular examples 28 to 33, wherein advancing the introducer relative to the sheath shaft comprises advancing the distal end of the second distal portion to a position proximal of a distal end of the sheath shaft.

Example 38: The method of any example herein, in particular example 37, wherein advancing the introducer relative to the sheath shaft comprises advancing the distal end of the second distal portion through both a portion of the shaft delivery lumen proximal of the expandable distal end portion and at least a portion of the shaft delivery lumen extending through the expandable distal end portion.

Example 39: The method any example herein, in particular examples 28 to 37, wherein providing the introducer comprises providing a distal end of the second distal portion of the introducer shaft frictionally fitted against a respective inner wall surface portion defining the shaft delivery lumen extending through the expandable distal end portion.

Example 40: The method of any example herein, in particular example 39, wherein the distal end of the second distal portion comprises a circumferential portion and wherein providing the distal end of the second distal portion of the introducer shaft frictionally fitted against a respective inner wall surface portion comprises providing the distal end of the second distal end portion frictionally fit against a respective circumferential inner wall surface portion defining the shaft delivery lumen.

Example 41: The method of any example herein, in particular examples 28 to 40, further comprising removing a spacer member from between a proximal end of a sheath hub and a distal end of an introducer hub prior to advancing the introducer relative to the shaft.

Example 42: The method of any example herein, in particular example 41, wherein removing the spacer member comprises removing the spacer member from around the introducer shaft.

Example 43: The method of any example herein, in particular example 41 or 42, wherein removing the spacer member comprises sliding a corresponding portion of the introducer shaft out from a recess extending through a longitudinal dimension of the spacer member.

Example 44: The method of any example herein, in particular examples 28 to 43 further comprising: retracting the introducer relative to the sheath shaft to remove the introducer; and advancing at least a portion of a delivery catheter carrying a medical device through the shaft delivery lumen while the expandable distal end portion is in the open state.

Example 45: The method of any example herein, in particular example 44, further comprises advancing the delivery catheter carrying the medical device to a heart.

Example 46: The method of any example herein, in particular examples 28 to 45, wherein inserting the at least a portion of the sheath shaft and introducer shaft into the bodily lumen comprises inserting the at least a portion of the sheath shaft and introducer shaft into a femoral artery.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for case of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical access system comprising:
an expandable access sheath comprising:
a sheath hub; and
a sheath shaft extending distally from the sheath hub and comprising an expandable distal end portion, the sheath shaft comprising a shaft delivery lumen extending therethrough; and
an introducer comprising an introducer shaft, the introducer shaft being configured to be at least partially disposed within the shaft delivery lumen while the expandable distal end portion is in a closed state, the introducer shaft comprising:
a first distal portion comprising a first diameter;
a second distal portion proximal of the first distal portion, the second distal portion comprising a second diameter larger than the first diameter; and
an externally oriented transition surface portion extending between a first externally oriented surface portion defining a portion of the first distal portion having the first diameter and a second externally oriented surface portion defining a portion of the second distal portion having the second diameter, the externally oriented transition surface portion and the second externally oriented surface portion forming an angle between 80 degrees and 100 degrees.

2. The system of claim 1, wherein the externally oriented transition surface portion is perpendicular to the second externally oriented surface portion.

3. The system of claim 1, wherein the first and second externally oriented surfaces are parallel to a longitudinal axis of the introducer.

4. The system of claim 1, wherein the first diameter and the second diameter have a ratio between 1:3 and 2:3.

5. The system of claim 1, wherein the introducer shaft further comprises a proximal portion proximal of the second distal portion, the proximal portion having a third diameter smaller than the second diameter.

6. The system of claim 1, wherein a remaining portion of the introducer shaft proximal of the second distal portion comprises the second diameter.

7. The system of claim 1, wherein, while the expandable distal end portion is in the closed state, a distal end of the second distal portion is configured to be positioned within the shaft delivery lumen at a position proximal of a proximal end of an inner wall surface portion defining the shaft delivery lumen extending through the expandable distal end portion.

8. The system of claim 1, wherein a distal end of the second distal portion frictionally fits against a respective inner wall surface portion defining the shaft delivery lumen extending through the expandable distal end portion.

9. The system of claim 8, wherein, while the expandable distal end portion is in the closed state, the distal end of the second distal portion is configured to be positioned against a proximal end or a proximal portion of the inner wall surface portion defining the shaft delivery lumen extending through the expandable distal end portion.

10. The system of claim 1, wherein a distal end of the second distal portion is a circumferential portion extending circumferentially around the introducer shaft.

11. The system of claim 1, further comprising a spacer member between a proximal end of the sheath hub and a distal end of an introducer hub, and wherein the spacer member comprises a mating portion, the mating portion having a recess extending through a longitudinal dimension, and the recess being configured to at least partially receive a corresponding portion of the introducer shaft.

12. A medical introducer comprising:
an introducer hub; and
an introducer shaft extending distally from the introducer hub, the introducer shaft comprising:
a first distal portion comprising a first externally oriented surface portion defining at least a portion of the first distal portion having a first diameter;
a second distal portion proximal of the first distal portion, the second distal portion comprising a second externally oriented surface portion defining at least a portion of the second distal portion having a second diameter that is larger than the first diameter; and
an externally oriented transition surface portion extending between the first and second externally oriented surface portions, the externally oriented transition surface portion being angled relative to the second externally oriented surface portion.

13. The introducer of claim 12, wherein the externally oriented transition surface portion is perpendicular to the second externally oriented surface portion.

14. The introducer of claim 12, wherein a remaining portion of the introducer shaft proximal of the second distal portion comprises the second diameter.

15. The introducer of claim 12, wherein a distal end of the second distal portion is disposed around a portion of a circumference of the introducer shaft.

16. A method of delivering a medical device, the method comprising:
providing an expandable access sheath comprising a sheath shaft that includes an expandable distal end portion in a closed state, the sheath shaft comprising a shaft delivery lumen extending therethrough; and
providing an introducer comprising an introducer shaft disposed at least partially within the shaft delivery lumen, the introducer shaft comprising an externally oriented transition surface portion extending between a first distal portion and a second distal portion, the second distal portion comprising a diameter larger than that of the first distal portion and being proximal of the first distal portion, the externally oriented transition surface portion and a second externally oriented surface portion of the second distal portion angled relative to one another;
inserting at least a portion of the sheath shaft and the introducer shaft into a bodily lumen; and
advancing the introducer relative to the sheath shaft and pushing a distal end of the second distal portion of the introducer shaft against an inner wall surface portion defining the shaft delivery lumen extending through the expandable distal end portion to thereby cause the expandable distal end portion to transform to an open state.

17. The method of claim 16, wherein pushing the distal end of the second distal portion against the inner wall surface portion comprises:
pushing the distal end against a proximal portion of the inner wall surface portion defining the shaft delivery lumen extending through the expandable distal end portion; or
pushing the distal end against a proximal end of the inner wall surface portion defining the shaft delivery lumen extending through the expandable distal end portion.

18. The method claim 16, wherein advancing the introducer relative to the sheath shaft comprises advancing the distal end of the second distal portion through a portion of the shaft delivery lumen proximal of the expandable distal end portion.

19. The method of claim 16, wherein advancing the introducer relative to the sheath shaft comprises advancing the distal end of the second distal portion to a position distal of a distal end of the sheath shaft or to a position at a distal end of the sheath shaft.

20. The method of claim 16, further comprising sliding a corresponding portion of the introducer shaft out from a recess extending through a longitudinal dimension of a spacer member prior to advancing the introducer relative to the sheath shaft.
